SEQUENCE LISTING (4 sequences)

SEQ ID NO: 1 - DNA, 1431 bp, Nicotiana benthamiana
  source 1..1431: mol_type = unassigned DNA
atgggtcagctccattttttcttctttcctatgatggctcagggccacatgattcctacactagacatggcgaagctcgttgcttcacgtggtgttaaggccactattatcacaaccccactcaatgaatctgttttctccaaatctattcaaagaaacaagcatttgggtatcgaaatcgaaatccgtttgatcaaattcccagctattgaaaatgacttacctgaagaatgcgaacgcctcgatcaaatcccttcagatgagaagctcccaaatttcttcaaagctacagctatgatgcaagaaccactggaacagcttattgaagaatgtcgccctaattgtcatgtttctgatatgttccttccttggactactgatagtgcagccaaatttaacatcccgagaatagttttccatggcacaagcttctttgctctttgcgttgagaatagcgtcaggctaaataagcctttcaagaatgtgtcctctgattctgaaacttttgttgtaccgaatttgcctcacgaaattaaactgaccagaacccaggtgtctccgtttgagcaatctggggaagagacaactatggcccggatgataaaaacagtctgggaatcagattcaaggagctatggagttgttttcaacagtttctatgagcttgaaacagattatgtggagcattatactaaggtgctgggtagaagagcttgggctattggcccgctatcgatgtgcaacagggacattgaagataaagctgaaagaggaaagaaatcctctattgataaacacgagtgcttgaaatggcttgattcgaagaaaccaagttccgtcgtttacatttgtttcggaagcgtagcgaatttcactgcatcacaactgcacgaactcgctatgggaattgaagcttctggacaagaattcatttgggttgttagaacagaactagacaacgaagattggttgcctgaaggatttgaagaaagaacaagagagaaaggattaataataagaggatgggcaccccaagtagtaattcttgatcatgaatctgtgggagcttttgttacacattgtggttggaattcaacactagaaggagtttcaggaggtgttccaatggtaacatggcctgtatttgccgagcaatttttcaatgagaagttagtgactgaggttttgagaactggggctgatgttggttcgattcagtggaaaagatcagctagtgaaggagtgaaaagagaagcaatagctaaggcaatacagagagtaatggtgagtgaagaagcagagggattcagaaacagagctaaagcgtataaggaaatggcaagaaaagctgttgaagaaggaggatcatcttacactggattgactactttgttggaagatataagtacatatagttccactggacattaa

SEQ ID NO: 2 - protein, 476 aa, Nicotiana benthamiana
MGQLHFFFFPMMAQGHMIPTLDMAKLVASRGVKATIITTPLNESVFSKSIQRNKHLGIEIEIRLIKFPAIENDLPEECERLDQIPSDEKLPNFFKATAMMQEPLEQLIEECRPNCHVSDMFLPWTTDSAAKFNIPRIVFHGTSFFALCVENSVRLNKPFKNVSSDSETFVVPNLPHEIKLTRTQVSPFEQSGEETTMARMIKTVWESDSRSYGVVFNSFYELETDYVEHYTKVLGRRAWAIGPLSMCNRDIEDKAERGKKSSIDKHECLKWLDSKKPSSVVYICFGSVANFTASQLHELAMGIEASGQEFIWVVRTELDNEDWLPEGFEERTREKGLIIRGWAPQVVILDHESVGAFVTHCGWNSTLEGVSGGVPMVTWPVFAEQFFNEKLVTEVLRTGADVGSIQWKRSASEGVKREAIAKAIQRVMVSEEAEGFRNRAKAYKEMARKAVEEGGSSYTGLTTLLEDISTYSSTGH

SEQ ID NO: 3 - DNA, 1464 bp, Catharanthus roseus
  source 1..1464: mol_type = unassigned DNA
atggttaatcagctccatattttcaacttcccattcatggcacagggccatatgttacccgccttagacatggccaatctattcacttctcgtggagtcaaagtaacattaatcacaacccatcaacatgttcccatgtttacaaaatccatagaaaggagcagaaattctggatttgatatatccattcaatccatcaaattcccagcttcagaagttggtttacctgaaggaatcgaaagtctagatcaagtttcaggggacgacgaaatgcttcctaagttcatgagaggagttaatttactccaacaacctctcgaacaactattgcaagaatctcgtcctcattgtcttctttctgatatgttcttcccttggactactgaatctgctgctaaatttggtattcccagattgctttttcatgggtcctgttcctttgccctctctgcagctgaaagtgtgagaagaaataaacctttcgagaatgtttccacagacacagaggaatttgttgtgcctgatcttccccaccaaattaaattaaccagaacacaaatttcaacatacgaaagggaaaatattgagtcagattttaccaaaatgctgaagaaagttagggattcagaatccacatcttacggagttgtagtcaatagtttctatgaacttgaaccagattatgccgattattacatcaacgttttgggaagaaaagcatggcatatagggccttttttgctttgtaacaaattacaagctgaagataaagcccaaagggggaagaaatcagcaattgatgcagacgaatgtttaaattggcttgattcgaaacaaccaaattccgtaatttatctctgtttcggaagtatggccaatttaaattctgcccaattacacgaaattgcaacagcccttgaatcctccggccaaaatttcatctgggttgttagaaaatgtgtggacgaagaaaacagttcaaaatggtttccagaaggattcgaagaaagaacaaaagaaaaagggctaattataaagggatgggcaccacaaaccctaattcttgaacacgaatcagtaggagcatttgttacccattgtggttggaattcaactcttgaaggaatctgcgcaggggttcctctggtgacttggcctttctttgctgagcaatttttcaatgagaaattgattacagaggtactgaaaacgggatacggagttggggctcggcaatggagtagagtttcaacagagattataaaaggagaagccatagctaatgctattaatcgagtaatggtgggtgatgaagctgttgagatgagaaacagagcaaaagatttgaaggaaaaggcaagaaaagctttggaagaagatggatcttcttatcgtgatcttactgctcttattgaagaattgggggcatatcgttctcaagttgaaagaaagcaacaagactag

SEQ ID NO: 4 - protein, 487 aa, Catharanthus roseus
MVNQLHIFNFPFMAQGHMLPALDMANLFTSRGVKVTLITTHQHVPMFTKSIERSRNSGFDISIQSIKFPASEVGLPEGIESLDQVSGDDEMLPKFMRGVNLLQQPLEQLLQESRPHCLLSDMFFPWTTESAAKFGIPRLLFHGSCSFALSAAESVRRNKPFENVSTDTEEFVVPDLPHQIKLTRTQISTYERENIESDFTKMLKKVRDSESTSYGVVVNSFYELEPDYADYYINVLGRKAWHIGPFLLCNKLQAEDKAQRGKKSAIDADECLNWLDSKQPNSVIYLCFGSMANLNSAQLHEIATALESSGQNFIWVVRKCVDEENSSKWFPEGFEERTKEKGLIIKGWAPQTLILEHESVGAFVTHCGWNSTLEGICAGVPLVTWPFFAEQFFNEKLITEVLKTGYGVGARQWSRVSTEIIKGEAIANAINRVMVGDEAVEMRNRAKDLKEKARKALEEDGSSYRDLTALIEELGAYRSQVERKQQD

(19) United States
(12) Patent Application Publication
(10) Pub. No.: US 2026/0366052 A1
(43) Pub. Date: Nov. 19, 2026

(54) NOVEL CANNABINOID-OLIGOSACCHARIDES

(71) Applicant: BIOSYNTH GMBH, Wien (AT)

(72) Inventors: Heimo Adamski, Langenpreising (DE); Thilo Fischer, Ismaning (DE); Fong-Chin HUANG, Freising (DE); Tobias VALLON, Heilbronn (DE)

(21) Appl. No.: 19/136,626

(22) PCT Filed: Dec. 6, 2023
(86) PCT No.: PCT/EP2023/084555

§ 371 Date: Jun. 6, 2025

(30) Foreign Application Priority Data
Dec. 8, 2022 (DE) 10 2022 004 596.6

(51) Int. Cl. C12P 19/44; A61K 31/702; C12P 19/18

(52) U.S. Cl. CPC C12P 19/44; A61K 31/702; C12P 19/18

(57) ABSTRACT

The present invention describes novel oligosaccharides of cannabidiol and other cannabinoids and methods for their production and uses thereof. The production comprises contacting cannabidiol with UDP-carbohydrate-dependent glycosyltransferase (UGT73A class) to produce cannabinoid-β-D-glycosides followed by contacting product with cyclomaltodextrin glucanotransferase and cyclodextrin to elongate sugar moieties with (1-4)-α-D-glucopyranosides to yield mixed β-α-structures in recombinant hosts with amphiphilic structures as surfactant solubilizer and has different enzymatic degradability of α-versus β-D-glucopyranosyl residues by amylases and beta-glucosidases allowing targeted drug delivery.

### FIELD OF THE INVENTION

The invention describes novel oligosaccharides of cannabidiol and other cannabinoids and special processes for their production, isolation, and applications which are facilitated by their specific characteristics.

### BACKGROUND OF THE INVENTION

Cannabinoids are secondary metabolites of hemp (Cannabis sativa) and are prenylated phenols. Major cannabinoid compounds are Δ⁹-tetrahydrocannabinol (THC), cannabidiol (CBD) (FIG. 1), cannabichromene (CBC), and cannabigerol (CBG). Cannabinol (CBN) is known from some cultivars of hemp and is derived from THC. These lead cannabinoid compounds vary in their respective relative ratios in different cultivars of hemp. Apart from neutral cannabinoids cannabinoid acids with carboxyl groups occur and are dominant in fresh plant material. Acidic cannabinoids are precursors of cannabinoids and can also non-enzymatically be converted to the later. Up to some 60 different cannabinoid compounds are known from hemp cultivars. Non-acidic cannabinoids are lipophilic compounds, often with phenolic structure and mainly being C₂₁ compounds. Parts of the structure arise from fatty acid and isoprenoid biosynthesis as well as polyketide pathway.

Various forms of cannabinoids are being consumed all over the world. This is either as an herb (marijuana) or in resin form (hash oil), by an estimated 2.6-5.0% of the world population (UNODC, 2012). Pharmaceutical products containing cannabinoid are available for medical use in several countries, either containing natural cannabis extracts (Sativex®) or the synthetic cannabinoids (dronabinol, nabilone). THC from Cannabis sativa has been approved by the Food and Drug Administration (FDA) for the control of nausea and of vomiting associated with chemotherapy and, more recently, for appetite stimulation of AIDS patients. In Germany, flowers of Cannabis (“Cannabis flos”) are pharmaceutically used, Dronabinol may by prescribed as a formulation with specific treatments. Other biological activities allow therapeutic applications like in the treatment of glaucoma, migraine headaches, spasticity, anxiety, and as an analgesic.

It is well documented that agents, such as cannabinoids and endocannabinoids that activate cannabinoid receptors in the body modulate appetite, and alleviate nausea, vomiting, pain (Martin B. R. and Wiley, J. L, Mechanism of action of cannabinoids: how it may lead to treatment of cachexia, emesis, and pain, Journal of Supportive Oncology 2:1-10, 2004), multiple sclerosis (Pertwee, R. G., Cannabinoids, and multiple sclerosis, Pharmacol. Ther. 95, 165-174, 2002), and epilepsy (Wallace, M. J., Blair, R. E., Falenski, K. W W., Martin, B. R., and DeLorenzo, R. J. Journal Pharmacology and Experimental Therapeutics, 307:129-137, 2003). In addition, CB2 receptor agonists have been shown to be effective in treating pain (Clayton N., Marshall F. H., Bountra C., O'Shaughnessy C. T., 2002. CB1 and CB2 cannabinoid receptors are implicated in inflammatory pain. 96, 253-260; Malan T. P., Ibrahim M. M., Vanderah T. W., Makriyannis A., Porreca F., 2002. Inhibition of pain responses by activation of CB (2) cannabinoid receptors. Chemistry and Physics of Lipids 121, 191-200; Malan T. P., Jr., Ibrahim M. M., Deng H., Liu Q., Mata H. P., Vanderah T., Porreca F., Makriyannis A., 2001. CB2 cannabinoid receptor-mediated peripheral antinociception. 93, 239-245.; Quartilho A., Mata H. P., Ibrahim M. M., Van derah T. W., Porreca F., Makriyannis A., Malan T. P., Jr., 2003. Inhibition of inflammatory hyperalgesia by activation of peripheral CB2 cannabinoid receptors. Anesthesiology 99, 955-960) and multiple sclerosis (Pertwee, R. G. Cannabinoids and multiple sclerosis, Pharmacol. Ther. 95, 165 174, 2002) in animal models.

Recently, approvement of cannabis and cannabinoid products for both recreational and medical uses has extended. Development of more efficient production and isolation of cannabinoid compounds is needed. Traditional methods of cannabinoid production typically focus on extraction and purification of cannabinoids from raw harvested Cannabis. However, traditional cannabinoid extraction and purification methods face technical and practical problems, primarily the need of non-aqueous solvents (organic solvents (U.S. Pat. No. 6,403,126 (Webster et al.); U.S. patent application No. 20160326130 (Lekhram et al.); butane) or supercritical CO2 (CA2424356 (Muller et al.)) for extraction and purification of the lipophilic cannabinoids. An analogous demand for water-soluble forms of cannabinoids occurs in their application, mainly due to instability (low shelf-life) and turbidity of emulsions of such water-based products.

Glycosides of cannabinoids have been studied. Tanaka et al. (Journal of Natural Products (1993) 56 (12): 2068-2072) described glucosides of cannabinol by biotransformation with in vitro plant tissue. Tanaka et al. (Plant Cell Reports (1996) 15:819-823) described 3-D-glucopyranosides of cannabidiol and cannabidiolic acid obtained by biotransformation with Pinellia ternata plant tissue.

US20190085347A1 disclosed “high level in vivo biosynthesis and isolation of water-soluble cannabinoids in plant systems”. Water solubility of cannabinoids is realized in a last biosynthetic step via glucosylation of cannabinoids by heterologous expression of suitable plant UDP-dependent glycosyl- or glucuronosyltransferases, which produce β-D-glucopyranosides or β-D-glucuronopyranosides, respectively.

WO2020239784A1 describes “genetically modified host cells producing glycosylated cannabinoids”, relying on UDP-dependent glycosyltransferases and, consequently, producing β-D-glycosides all over, except one example of an α-L-rhamnoside, the rare representative of L-carbohydrates.

WO2021146687A1 claims “a cannaboside composition and method to produce”; this is by feeding to animals (especially insects/Orthoptera, crickets). Their description comprises β-D-glucopyranosides of cannabinoid compounds obtained with up to six glucosidic residues.

WO2021173130A1 discloses “novel cannabinoid glycosides and uses thereof”. Cannabinoid glucopyranoside structures given are all β-anomers with up to four glucopyranoside residues.

WO2022099078A1 describes the “production of glycosylated cannabinoids” via UDP-dependent glycosyltransferases from Arabidopsis thaliana and Helianthus annuus.

WO2022126028A1 reports on a “continuous enzymatic perfusion reactor system” to produce cannabinoid glycosides.

Summarizing, all reports focus on producing and/or handling β-D-glycosides, mostly β-D-glucopyranosides of cannabinoids with mainly one to four carbohydrate units and various inter-carbohydrate links (1-3, 1-4, 1-6).

A general and important need for cannabinoid glycosides is their degradability by enzymatic hydrolysis, like with widespread β-glucosidases, after consumption of the cannabinoid containing product.

### Object of the Invention

The task of the invention was to find an efficient process for production of highly glucosylated cannabidiol derivates which would have superior water solubility, but also with a structure typical for surfactants/solubilizers, designed to allow solubilization or emulsifying of lesser glycosylated or free cannabinoids and also with a structure allowing for enzymatic cleavage. This would allow development of superior processes of isolation and would facilitate new and improved forms of application of cannabinoids. This would be exemplified with cannabidiol as a lead cannabinoid beyond THC.

The invention had to solve the problem of producing such highly glucosylated cannabinoid derivates, which should also be degradable by enzymes with human consumption. Beyond state of the art, the requirement for water soluble cannabinoid derivates, however, would also be fulfilled by α-D-glucopyranosides of cannabinoids. Furthermore, α-D-glucopyranosides can be hydrolyzed by amylases, typical enzymes of the human digestive tract. Aiming at very good water solubility and at a structure typical for surfactants/solubilizers, a production method for highly glucosylated cannabinoid derivates had to be invented.

Cyclomaltodextringlucanotransferases are a well-known enzyme class. They not only produce α-, β-, γ-(6-, 7-, 8-membered rings) or higher cyclodextrins from starch, but also to perform transgycosylation reactions with carbohydrate accpetors and also hydrolysis with water as acceptor (https://www.cazypedia.org/index.php/Glycoside_Hydrolase_Family_13). However, this is mainly reported for transfer to other carbohydrates/oligosaccharides/polysaccharides, with only minor activities reported for direct transglycosylation of non-carbohydrate acceptor substrates. Unlike UDP-α-glucose-dependent glycosyltransferases stereochemistry at the carbohydrate is not converted by the substitution mechanism to build-glucopyranosides but retained by a double substitution with an enzyme-bound intermediate. Consequently, with (1-4)-α-D-glucopyranosyl residues as a co-substrate like from cyclodextrines α-D-glucopyranosides are formed by cyclomaltodextringlucanotransferases. Some reactions with these enzymes like partial transglycosylation, partial re-transglycosylation (disproportionation), or partial hydrolysis (e.g. Ara et al. Glycobiology, 2015, vol. 25, no. 5, 514-523,doi: 10.1093/glycob/cwu182) can lead to a lesser number of (1-4)-α-D-glucopyranosyl residues than expected by simple cyclodextrin-residue-transfer. Such lesser numbers of α-D-glucopyranosyl residues can also be obtained from transglycosylation reactions by glucansucrases (https://www.cazypedia.org/index.php/Glycoside_Hydrolase_Family_70) which use disaccharides, mainly sucrose, instead of cyclodextrines as a co-substrate.

Transglycosylation of natural glucosides as substrates has been shown with steviol glycosides and rebaudioside A and been found to improve taste quality of the derived compounds (Muñoz-Labrador et al. Foods 2020, 9, 1753; doi: 10.3390/foods9121753). Given this substrate acceptance and the ones of UDP-dependent glycosyltransferases (once an enzyme with a suitable specificity for the aglycon has been identified), a possible strategy may be projecting highly glycosylated cannabinoid derivatives via an initial and direct stem glycosylation made of β-D-glucopyranosides by UDP-dependent glycosyltransferases and a subsequent and stepwise transfer of six-to-eight units (by cyclomaltodextringlucanotransferases) or of single (by glucansucrases) (1-4) α-D-glucopyranosides residues. Glucansucrases from Liquorilactobacillus nagelii and Liquorilactobacillus hordei used for the invention are known to be dextransucrases (Bechtner et al. 2022, Gels 2022, 8, 171. https://doi.org/10.3390/gels8030171). This strategy would allow a formation of cannabinoid-oligosaccharides with the intended mixed β/α-structure.

### SUMMARY OF THE INVENTION

The invention is involved with processes for production and isolation of cannabinoid-oligosaccharides, demonstrated by cannabidiol-oligosaccharides, which are based on the use of specific plant UDP-glucose-dependent glycosyltransferases of the class UGT73a in a first step and of cyclomaltodextringlucanotransferase(s) and/or glucansucrases in a second one to allow formation of a cannabinoid-oligosaccharide with an amphiphilic structure typical for water soluble compounds and for emulsifiers/solubilizers. Preferred embodiments are processes, which are based on the heterologous expression in microorganisms like E. coli or in Cannabis sativa; processes relying on heterologous expression in Cannabis sativa may use the glucosyl residues as protective groups for preventing tetrahydrocannabinol-biosynthesis and give the option of aqueous extraction as a first step of production. Other subjects of the invention are compositions, which are obtained by these processes, processes for isolation of cannabidiol-oligosaccharides from such compositions and the use of these cannabinoid-oligosaccharides as active principles. Also concentrated solutions of active principles, as medicine or as veterinary medicine are an advantageous embodiment of the invention. Glycosidases, especially β-glucosidases, and amylases can be applied with cannabinoid-oligosaccharides in various forms to trigger aglycon release. The differences in the enzymatic degradability of α-versus β-D-glucopyranosyl residues in cannabinoid-oligosaccharides by amylases and β-glucosidases provide allow targeted drug delivery.

More specifically, the present invention relates to the following:
(1) A method for producing cannabinoid-oligosaccharide(s), for example cannabidiol (CBD)-oligosaccharide(s), comprising the step of contacting a cannabinoid like CBD with an isolated UDP-carbohydrate-dependent glycosyltransferase preferably of the class UGT73A, preferably with the glycosyltransferases identified as [Seq1 (DNA), Seq2 (AA)=NbGT-fc6] and [Seq3 (DNA), Seq4 (AA)=CaGT2], or with UDP-carbohydrate-dependent glycosyltransferases, which show or a nucleic acid sequence identity of at least 70%, of at least 80%, of at least 90%, of at least 95%, of at least 98% to [Seq1=CaGT2], or with an amino acid sequence identity of at least 70%, of at least 80% of at least 90%, of at least 95% to [Seq2=CaGT2], in a reaction mixture under conditions, including any co-substrates and co-factors necessary for glucosyltransferase activity, effective to produce cannabinoid-glycoside(s), and of contacting this, subsequently or in parallel, or cannabinoid-β-D-glycosides from other sources, with a cyclomaltodextringlucanotransferase with at least one of its substrates cyclodextrin or substituted cyclodextrin (for example hydroxypropyl-, methyl-), linear maltodextrins, starch, or glycogen and another step of purifying the cannabinoid-oligosaccharide(s) from the reaction mixture to obtain the isolated cannabinoid-oligosaccharide(s).
(2) The method of (1), wherein the isolated UDP-carbohydrate-dependent glycosyltransferase uses UDP-glucose or an activated form of fructose, glucose, galactose, mannose, rhamnose, ribose, arabinose, xylose, or an activated oligosaccharide, or an activated aldonic or uronic acid, for example gluconic acid, glucuronic acid or galacturonic acid, or N-substituted derivatives of said carbohydrates, for example 2-N-acetyl glucosamine, as co-substrate and a cannabinoid like CBD, and wherein a cyclomaltodextringlucanotransferase uses the intermediary product (or added educt from other sources) cannabinoid-β-D-glycoside as a substrate to form higher glycosylated oligosaccharides (the latter with α-anomeric glucopyranosyl residues) and the products are the respective cannabidiol-oligosaccharides with stem β-D-glycosidic structures and branching α-glucopyranosyl-oligomers.
(3) The method of (1) or (2) wherein the isolated UDP-carbohydrate-dependent glycosyltransferase and the cyclomaltodextringlucanotransferase are obtained by heterologous expression in one or two organisms of species different from the origin species of the glycosyltransferase or the cyclomaltodextringlucanotransferase; preferably the conversion of cannabinoids to cannabinoid-oligosaccharide(s) relies on the cells supply of the co-substrate identified as UDP-glucose or as other activated carbohydrates and putatively on the cells supply or import of at least one of the substrates cyclodextrin or substituted cyclodextrin (for example hydroxypropyl-, methyl-), linear maltodextrins, starch, or glycogen, and takes place within the same heterologous organism(s), which may be optimized by genetic and/or gene technological means for the purpose.
(4) The method of any one of (1) to (3) wherein instead of or in addition to cyclomaltodextringlucanotransferase and its substrates (cyclodextrin or substituted cyclodextrin (for example hydroxypropyl-, methyl-), linear maltodextrins, starch, or glycogen) glucansucrase(s) and their substrate sucrose (or other compatible disaccharides) are used for the addition of (1-2)-, (1-3)-, (1-4)—, or (1-6)-linked α-D-glucopyranoside residues to produce staggered cannabinoid-oligosaccharides.
(5) The method of any one of (1) to (4) wherein cannabinoid-oligosaccharides can be obtained, for example but not limiting wherein CBD-2ßG-8αG and/or higher reaction products or staggered cannabinoid-oligosaccharides are obtained from cannabidiol with GT40/UDP-glucose and with cyclomaltodextringlucanotransferase/γ-cyclodextrin or with glucansucrases and sucrose (or other disaccharides).
(6) Cannabinoid-oligosaccharides obtained by processes according to any one of (1) to (5) with a glycosylation pattern comprising none or one to four β-D-glycopyranosyl stem residue(s) and one or more additional ((1-4)-α-D-glucopyranosyl)_{5,6, 7, or >7} (1-4)-α-D-glucopyranoside) n oligosaccharide residues or staggered cannabinoid-oligosaccharides, the staggered cannabinoid-oligosaccharides also be derived by other glycosyltransferase or transglycosylation reactions than cyclomaltodextringlucanotransferase like with glucansucrases and in this case may be not only be (1-4)-but also (1-2)-, (1-3)-, or (1-6)-linked, the (1-4)-α-D-glucopyranoside residue(s) may be directly attached to the cannabinoid, to its β-D-glycopyranosyl residue(s), or, if more than one, may also be attached to each other.
(7) CBD-oligosaccharides obtained according to (6) comprising none or one to four β-D-glucopyranosyl stem residue(s) and one or more additional ((1-4)-α-D-glucopyranosyl)_{5, 6, 7, or >7} (1-4)-α-D-glucopyranoside)ₙ oligosaccharide residues or being staggered cannabinoid-oligosaccharides which can be (1-2)-, (1-3)—, (1-4)-, or (1-6)-α-linked.
(8) The compounds CBD-2βG-6αG, CBD-(βG-6αG)₂, CBD-2βG-12αG, CBD-2βG-7αG, CBD-(βG-7αG)₂, CBD-2βG-14αG, CBD-2βG-8αG, CBD-(βG-8αG) 2, CBD-2βG-16αG, CBD-2βG-(6/7/8αG)ₙ, and staggered cannabinoid-oligosaccharides, the latter can be (1-2)-, (1-3)-, (1-4)-, or (1-6)-α-linked, or esters thereof, or acetal, or semiacetal derivatives thereof or an ester of the latter.
(9) The method of any one of (1) to (5) wherein strains of Escherichia coli or yeasts, for example Saccharomyces cerevisiae or Pichia pastoris, are used as microorganisms for production.
(10) The method of any one of (1) to (5) wherein the UDP-carbohydrate-dependent glycosyltransferase and the cyclomaltodextringlucanotransferase and/or a glucansucrase are heterologously expressed in Cannabis sativa and cannabinoids like cannabidiol from the secondary metabolism of the plant are used as substrate to be converted to the respective cannabinoid-oligosaccharides and these can be obtained by this means.
(11) The method of any one of (1) to (5) and (9) wherein the UDP-carbohydrate-dependent glycosyltransferase is heterologously expressed in Cannabis sativa and wherein the the glucose residues at the 2′ and 6′ hydroxy groups of a cannabinoid like cannabigerolic acid act as protective groups preventing or reducing metabolization and cyclization to tetrahydrocannabinol(s).
(12) Compositions obtained by methods of any one of (1) to (11), comprising
   cannabinoid-oligosaccharides, for example cannabidiol-oligosaccharides,
   components of the reaction medium according to (1) to (3), or
   components of an organism according to (3), (9), (10), (11), and/or
   components of a culture medium according to (3), (9) and their degradation products;
the compositions optionally may contain non-converted cannabinoid substrates like cannabidiol and byproducts, derived from cannabinoid-oligosaccharides, for example acetates with variable sites of substitutions at the glucopyranosylj residues.
(13) Further method by which cannabinoid-oligosaccharides obtained by method of any one of (1) to (11) are enriched and purified in a second step from resulting compositions according to (12) and wherein extractions with organic solvents, preferably ethylacetate, as well as adsorption to a lipophilic solid phase, preferably at a polystyrol-matrix, are used for enrichment and purification and an organic solvent, preferably methanol, is used for elution from this matrix.
(14) Method of anyone of (1) to (8) and (10) to (13) wherein the first step of obtaining the cannabinoid-oligosaccharides from the organism Cannabis sativa for subsequent enrichment and purification is an extraction with an aqueous phase, or a hot aqueous phase (water or steam), or a polar solvent, or a polar solvent mixture.
(15) Kit according to (1) to (14) for production, purification, and analytics of cannabinoid-oligosaccharides, for example cannabidiol-oligosaccharides, comprising the gene and/or the enzyme of [Seq 1] and a gene and/or enzyme of a cyclomaltodextringlucanotransferase and/or a glucansucrase in form of a DNA-sequence, or a codon-optimized DNA-sequence, or an amino acid sequence, or an isolated DNA, or a DNA in a vector or an expression vector, or the enzyme or a composition containing the enzyme, or a transgenic microorganisms or a transgenic plant, as well as specific means and instructions for performing the processes of production and purification and for analytics and for quality control of the products.
(16) Cannabinoid-oligosaccharides according to any one of (1) to (14), for example cannabidiol-oligosaccharides, optionally substituted at glycosidic residues, for example acetylated or peracetylated, for use as biologically active compounds, as medicine or veterinary medicine, which in comparison to the respective free cannabinoid can have an improved water solubility, can have a solubilizing or emulsifying effect on other glycosides of cannabidiol or other cannabinoids with lesser number of carbohydrate residues (for example CBD-1βG, CBD-2βG) or on free cannabidiol or on other free cannabinoids, or on other components with low solubility within a composition, for example on aroma compounds or other biologically active compounds in a beverage, a different taste or mouth feeling, a different uptake to the body, a changed metabolization, a changed activity, or a changed pharmacokinetic.
(17) Composition comprising
   Cannabinoid-oligosaccharides, for example cannabidiol-oligosaccharides; and
   at least one glycosidase, preferably at least one ß-glucosidase, or a composition of glycosidases, preferably ß-glucosidases; and/or
   microorganisms which produce glycosidases, preferably ß-glucosidases; and/or
   amylase(s) and/or
   encapsulated forms of single, some, or all said components of the composition
for use as active pharmaceutical principle, medicine, or veterinary medicine.
(18) A method using cannabinoid-oligosaccharides, for example cannabidiol-oligosaccharides and/or substituted derivatives according to claim 16 or compositions with these according to (17) in concentrated aqueous solution to obtain a concentrate, hereby obtaining an intermediate product for the purposes described in (16) and (17), which can be used advantageously for the production of the respective end product, which as such concentrates are more durable or by their lesser volume and lesser weight are better storable or transportable; such a concentrate shows an at least twofold concentration of cannabinoid-oligosaccharides or their derivatives in comparison to the concentration in the respective end product (for example a beverage), more preferred are at least fivefold concentrations in the said concentrate.
(19) The use of cannabinoid-oligosaccharides with mixed α- and β-glucopyranosyl structure for differential release of the aglyca relying on the organ-specific (for example mouth cavity, stomach, small and large intestine) presence of hydrolytic enzymes/enzymes with glycosidase activity (for example various human amylases, for example microbial β-glucosidases) for the purpose of targeted drug delivery.

### BRIEF DESCRIPTION OF THE FIGURES

FIG. 1: Chemical structure of cannabidiol (top) Note that positions 2′ and 6′ are equivalent by rotation around the resorcine-cyclohexene link. Chemical structure of CBD-2βG (bottom) (“β-stem glucosylation”).

FIG. 2: Chemical structure of the cannabinoid-oligosaccharide cannabidiol-2′-β-D-glucopyranosyl-((1-4)-α-D-glucopyranosyl)) 7-(1-4)-α-D-glucopyranoside, 6′-β-D-glucopyranoside (CBD-2β-8αG), a pivotal cannabinoid-oligosaccharide obtained via cannabidiol-2′6′-di-β-D-glucopyranoside by reaction with γ-cyclodextrin catalyzed by cyclomaltodextringlucanotransferase. It consists of a 2′,6′-β-stem glucosylation and a modular α-glucosylation by chains of 6, 7 or 8 or >8 α-glucopyranosyl residues, 8 in this example (n=7, n+1 for the end glucopyranosyl group). Lower numbers of (1-4)-α-D-glucopyranoside residues are commonly observed due to previously reported disproportionation and hydrolysis activities of cyclomaltodextringlucanotransferase (“staggered cannabinoid oligosaccharides”). R¹ and R² can also each be such modular α-glucopyranosyl chains. The modular α-glucopyranosyl chains will have a helical structure like with starch. Cannabinoid oligosaccharides obtained with glucansucrase can possess other links than (1-4)-α, like with dextransucrases of Example 6 which give (1-6)-α-links.

FIG. 3: GT73A-Gene (NbGT-fc6; Seq1 (DNA) (=SEQ ID NO:1), see FIG. 3A; Seq2 (AA) (=SEQ ID NO:2), see FIG. 3B), which was used in Example 1 for heterologous expression and biotransformation of cannabidiol to CBD-2βG, and second gene GT73A-Gene (CaGT2; Seq3 (DNA) (=SEQ ID NO: 3), see FIG. 3C; Seq4 (AA) (=SEQ ID NO:4), see FIG. 3D, from Catharanthus roseus) which also allows production of CBD-2βG.

FIG. 4: Synthesis of CBD-oligosaccharides from CBD-2βG (Example 1), analysis of aliquots by HPLC. Enzymatic conversion of CBD-2βG with CGTase and (graphics top to bottom) α-, β-, γ-cyclodextrin, and starch as co-substrates, and control with only CBD-2βG. Products are observed with all co-substrates. The peak eluting before the CBD-2βG peak (4.2 min) is CBD-2βG-1αG (3.9 min). The peaks eluting before this (at about 3.6 min) are CBD-2βG-2αG and CBD-2βG-3αG (minor peak at about 3.4 min).

FIG. 5: Synthesis of staggered CBD-oligosaccharides via CBD-2βG (Example 2), analysis of aliquots by HPLC. Bottom curve: control: CBD-2βG and γ-cyclodextrin substrates, reaction assay without CGTase enzyme. Top curve: The formed peaks eluting before CBD-2βG (at about 4.1 min) are CBD-2βG-1αG (at about 3.9 min), CBD-2βG-2αG (at about 3.6 min) and CBD-2βG-3αG (minor peak at about 3.4 min), representing partial products of the projected CBD2βG-8αG.

FIG. 6: LC-MS analysis of staggered CBD-oligosaccharides (Example 3). Top graphic (FIG. 6a): Top box: Total ion current chromatogram. Lower boxes: chromatograms of individual compounds at their respective m/z in negative mode, CBD-2βG m/z 683 [M+HCOOH—H]⁻ (adduct with formic acid from solvent) (second box), CBD-2βG-1αG m/z 799 [M−H]⁻ (third box), CBD-2βG-2αG m/z 961 [M−H]⁻ (forth box), CBD-2βG-3αG m/z 1123 [M−H]⁻ (fifth box), respectively. Bottom graphic (FIG. 6b): MS/MS2 spectra of CBD-2βG m/z 683 [M+HCOOH—H]⁻ (first box), CBD-2βG-1αG m/z 799 [M−H]⁻ (second box), CBD-2βG-2αG m/z 961 [M−H]⁻ (third box), CBD-2βG-3αG m/z 1123 [M−H]⁻ (forth box). The MS2 spectra of all compounds show the expected fragmentation with loss on one or multiple glucopyranosyl residues (times (−162 D)).

FIG. 7: Hydrolytic cleavage of staggered CBD-oligosaccharides CBD-2βG-1αG, CBD-2βG-2αG, and CBD-2βG-3αG composition by saliva (top graphic, Example 4) or ß-glucosidase (middle graphic, Example 5) and control without hydrolyzing enzyme (substrate of the shown reaction, bottom graphic). The CBD-2βG peak elutes at 4.2 min, the peaks eluting before are CBD-2βG-1αG (3.9 min), CBD-2βG-2αG, (3.6 min), and CBD-2βG-3αG (minor amounts eluting little before). Products of hydrolyses are CBD-2βG and CBD-1βG (5.5 min).

FIG. 8: HPLC-analysis of formation of staggered CBD-oligosaccharides from CBD-2βG by glucansucrases of Lactobacillus hordei TM 1.1822 (top box) and Lactobacillus nagelii TM 1.1827 (bottom box) (Example 6). Curves are each for control (0), 1, 2 and 4 hours of incubation of the enzymatic reaction, from bottom to top. The substrate CBD-2βG elutes at about 4.2 min, the products CBD-2βG-1αG, CBD-2βG-2αG, and CBD-2βG-3αG (all with (1-6)-α-links) elute before this with decreasing elution times.

### DETAILED DESCRIPTION OF THE INVENTION

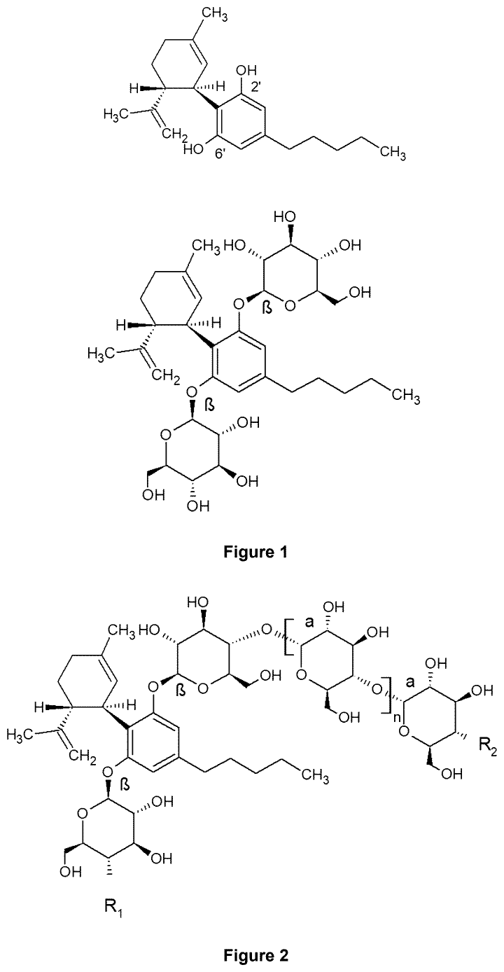

Surprisingly, it was found that cannabinoids like cannabidiol (FIG. 1 top) with only low numbers of β-D-glucopyranosyl-residues, for example CBD-1βG or CBD-2βG (FIG. 1 bottom), are very good substrates for cyclodextringlucanotransferases.

CBD-2βG was chosen for demonstration since it carries β-D-glucopyranosyl-residues at both of its hydroxy functions and hence is the simplest representative structure of this kind. This allows to transfer specifically groups of six, seven or eight (or higher number) of (1-4)-α-D-glucopyranosyl residues from either cyclodextrins, substituted cyclodextrins (like hydroxypropyl-, methyl-) or a mixture of such residues from linear substrate with polymeric (1-4)-α-D-glucopyranosyl-structure like starch or glycogen (FIG. 2).

Hence, highly glycosylated forms of cannabinoids are to be obtained, possessing an inherent and intended amphiphilic structure typical for surfactants/solubilizers and of mixed anomeric structure (β/α). For such, not only very good water solubility can be expected but also solubilizing effects on compounds with lesser water solubility like cannabinoids or cannabinoids with lower glycosylation pattern but also a variation in the mechanisms of aglycon release.

Like the β-D-glucopyranosyl-residues with β-D-glucosidases, α-D-glucopyranosyl residues can be cleaved off by the activity of amylases found in the human digestive tract (salivary, small intestine), allowing for a stepwise and putatively more targeted degradation and release of the aglycon, respectively, for the purpose of targeted drug delivery.

A positive effect on taste profile of products also seems achievable. Such derivatives with long hydrophilic residues (like well-known polysorbates) have optimal characteristics for application in pharmacy and other areas for diverse water-based products.

### DEFINITIONS

For the description of the invention the terms are understood like given below:

### Amylase

Specific glycosidases hydrolyzing (1-4)-α-D-oligo- or polysaccharides like starch or compounds with (1-4)-α-linked-oligosaccharide residues. The Greek prefix refers to enzyme subclasses, not to anomers of glycosides. α-amylases cleave randomly and produce dextrins ((1-4) α-linked-oligosaccharides), maltotriose (trisaccharide units of three 1-4-linked α-D-glucose molecules, maltose (disaccharide units of two 1-4-linked α-D-glucose molecules), and glucose. ß-amylases terminally cleave off maltose, and γ-amylases terminally cleave off glucose monosaccharide units. α-amylases occur in animals (salivary glands, pancreas), plants, microbes. ß-amylases occur in plants (seeds, fruits) and microbes. γ-amylases occur in animals (small intestine) and microbes.

### Anomers

Diastereomers of carbohydrates, here especially of D-glucose, referring to the acetalic hydroxy function at carbon atom 1 in a glucoside, corresponding to the α- or ß-form of the glucoside.

### Amphiphilic

Molecules with hydrophilic as well as lipophilic parts are called amphiphilic.

### Cannabidiol

2-[(1R,6R)-3-Methyl-6-prop-1-en-2-yl-1-cyclohex-2-enyl]-5-pentylbenzo-1,3-diol

### Cannabinoids

As used here, it refers to secondary metabolites specific of hemp (Cannabis sativa) which are prenylated phenols.

### Cannabinoid-Oligosaccharides

As used here the term refers to cannabinoids with a pattern of an initial stem of zero to four β-D-glucopyranosidyl residues and one or more additional ((1-4)-α-D-glucopyranosyl)_{5, 6, 7 or >7} (1-4)-α-D-glucopyranoside oligosaccharide residues. A pivotal exemplary structure is given in FIG. 2.

### Staggered Cannabinoid-Oligosaccharides

Cannabinoid-oligosaccharides can possess lesser numbers of (1-4)-α-D-glucopyranosyl residues than expected by simple cyclodextrin-residue-transfer by partial transglycosylation, partial re-transglycosylation (disproportionation), or partial hydrolysis. They can also be obtained by the use of glucansucrase enzymes and disaccharides like sucrose as a co-substrate and in this case can not only be (1-4)-α-linked, but also be (1-2)-, (1-3)-, or (1-6)-α-linked.

### Cyclomaltodextringlucanotransferase

Transferases transferring ((1-4)-α-D-glucopyranosyl)_{5, 6, 7 or >7} (1-4)-α-D-glucopyranoside oligosaccharides from α-, ß-, γ- or higher cyclodextrins (Greek prefix here refers to the type of cyclodextrin) by ring opening or from starch by helix opening. Definition follows https://www.cazypedia.org/index.php/Glycoside_Hydrolase_Family_13.

### Glycoside

Chemical structure formed by a carbohydrate with an alcohol, an amine, or a thiole (corresponding to O-, N- or S-glycosides). Only O-glycosides are considered here.

### Glucansucrase

Family GH70 glucansucrase (GS) enzymes catalyze the synthesis of α-glucans from sucrose. Definition follows

https://www.cazypedia.org/index.php/Glycoside_Hydrolase_Family_7.

### Glucansucrases

Glucansucrases can form alterans α-(1-3), (1-6), dextrans α-(1-6), reuterans α-(1-4), or mutans α-(1-3).

### Glucoside

Specific glycoside with glucose as the bound carbohydrate. As with glycosides α- and ß-D-glucosides are distinguished, dependent on the position of the oxygen atom forming the glycosidic bond in relation to the carbohydrate in standard projection (α-glucoside: oxygen upwards in relation to ring, in ß-glucosides downwards) (other definitions refer to configuration of C5 in relation).

### Glykosidase

Enzyme with a hydrolytic activity for glycosides, this is a cleavage of glycosides with uptake of a molecule of water. ß-Glycosidases can cleave ß-glycosides like ß-glucosides. Glucosidases are special glycosidases or specific activities of such.

### Lipophil

With affinity to fat or oil, respectively. The term describes molecules with low polarity.

### Transglycosylation

The transfer of carbohydrate residue from one glycoside to another.

### EXAMPLES

### Specific Chemicals Used

ß-Amylase was obtained from Roth (8717.1), Germany. Cyclomaltodextringlucanotransferase (CGTase) was obtained from Amano, Japan. Glucansucrases were directly obtained from cultures of Lactobacillus sp. Rapidase (Mixture of four β-glucosidases) was obtained from DSM Food specialties B.V., Netherlands, with the trade name Rapidase AR 2000.

### ABBREVIATIONS

Abbreviations used within the present invention are:

### CBD Cannabidiol

CBD-1βG Cannabidiol-2′-ß-D-glucopyranoside

CBD-2βG Cannabidiol-2′,6′-ß-D-diglucopyranoside

CBD-2βG-6αG Cannabidiol-2′-β-D-glucopyranosyl-((1-4)-α-D-glucopyranosyl))₅-(1-4)-α-D-glucopyranoside,6′-β-D-glucopyranoside

CBD-(βG-6αG)₂ Cannabidiol-2′-3-D-glucopyranosyl-((1-4)-α-D-glucopyranosyl))₅-(1-4)-α-D-glucopyranoside,6′-β-D-glucopyranosyl-((1-4)-α-D-glucopyranosyl))₅-(1-4)-α-D-glucopyranoside

CBD-2βG-12αG Cannabidiol-2′-β-D-glucopyranosyl-((1-4)-α-D-glucopyranosyl))₁₁-(1-4)-α-D-glucopyranoside,6′-β-D-glucopyranoside

CBD-2βG-7αG Cannabidiol-2′-β-D-glucopyranosyl-((1-4)-α-D-glucopyranosyl))₆-(1-4)-α-D-glucopyranoside,6′-β-D-glucopyranoside

CBD-(βG-7αG)₂ Cannabidiol-2′-β-D-glucopyranosyl-((1-4)-α-D-glucopyranosyl))₆-(1-4)-α-D-glucopyranoside,6′-β-D-glucopyranosyl-((1-4)-α-D-glucopyranosyl))₆-(1-4)-α-D-glucopyranoside

CBD-2βG-14αG Cannabidiol-2′-β-D-glucopyranosyl-((1-4)-α-D-glucopyranosyl))₁₃-(1-4)-α-D-glucopyranoside,6′-β-D-glucopyranoside

CBD-2βG-8αG Cannabidiol-2′-β-D-glucopyranosyl-((1-4)-α-D-glucopyranosyl))₇-(1-4)-α-D-glucopyranoside,6′-β-D-glucopyranoside (FIG. 2)

CBD-(βG-8αG) 2 Cannabidiol-2′-β-D-glucopyranosyl-((1-4)-α-D-glucopyranosyl))₇-(1-4)-α-D-glucopyranoside,6′-β-D-glucopyranosyl-((1-4)-α-D-glucopyranosyl))₇-(1-4)-α-D-glucopyranoside

CBD-2βG-16αG Cannabidiol-2′-β-D-glucopyranosyl-((1-4)-α-D-glucopyranosyl))₁₅-(1-4)-α-D-glucopyranoside,6′-β-D-glucopyranoside

CBD-2βG-(6/7/8αG)ₙ Cannabidiol-2′-β-D-glucopyranosyl-((1-4)-α-D-glucopyranosyl))_{5,6,7}-(1-4)-α-D-glucopyranoside,6′-ß-D-glucopyranoside (mixture of compounds with variable chain length obtained by use of starch, glycogen, dextrins, or mixtures of cyclodextrins as substrates), including compounds with multiple (n) such substituents in various positions.
CGTase Cyclomaltodextringlucanotransferase
GT Glycosyltransferase
mg milligram
min minute
mL milliliter
mM millimolar
rpm rotations per minute, speed of centrifugation
w/V weight/volume; concentration value

### Example 1

Formation of CBD-Oligosaccharides via CBD-2βG with Cyclomaltodextringlucanotransferase

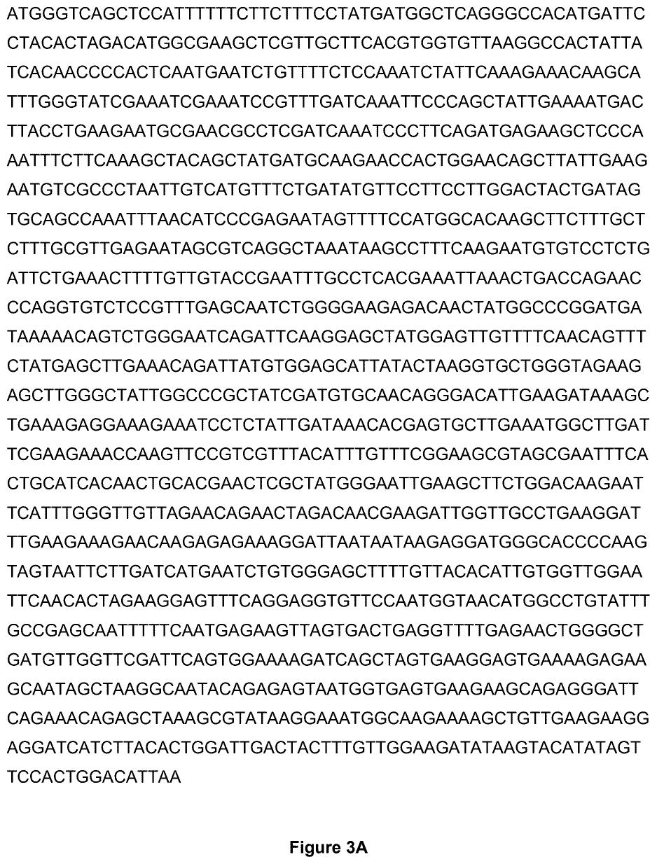

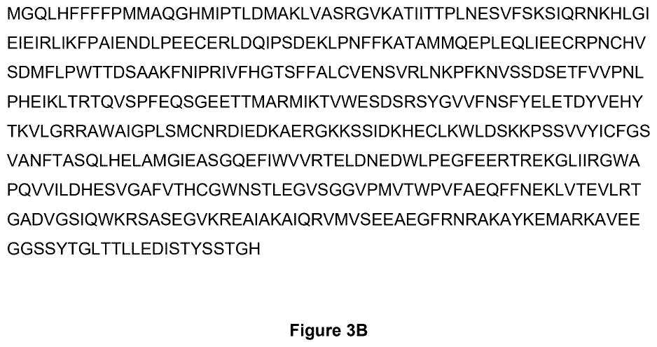

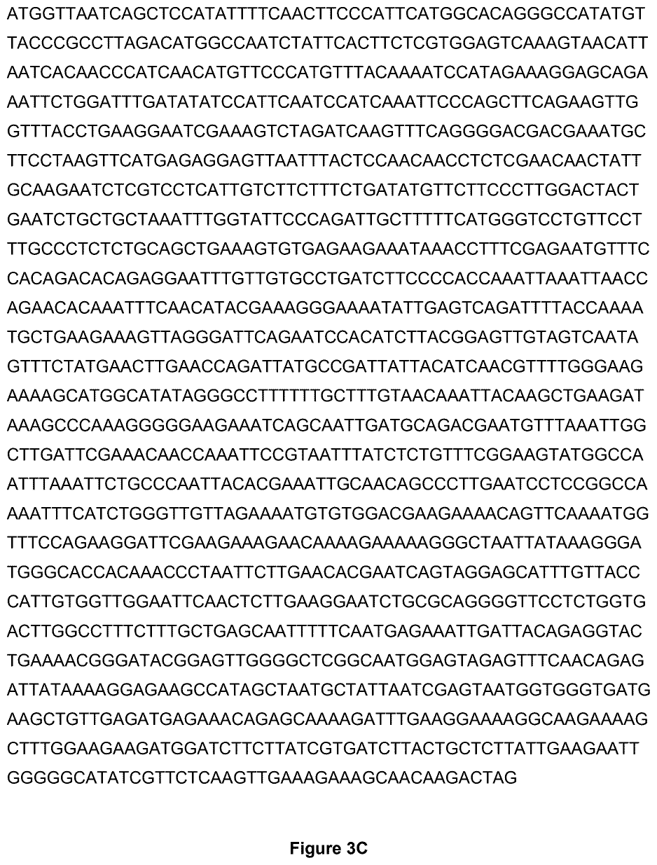

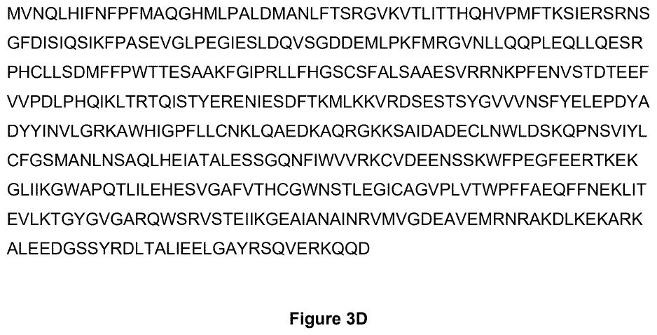

The glycosyltransferase UGT73A gene from Nicotiana benthamiana (sequence 1 (FIG. 3)) in an expression vector was used for the glucosylation of cannabidiol to CBD-2βG by biotransformation in E. coli BL21 (DE3) pLysS (Novagen, Schwalbach, Germany). Preparatory cultures were grown over night at 37 °C in Luria Bertani (LB)-medium containing 100 μg/mL ampicillin and 23 μg/mL chloramphenicol.

Next day 50 mL M9-minimal medium with 1% glucose and the said antibiotics were inoculated with 1 mL of the preparatory cultures and were cultivated at 37 °C and 160 rpm until an OD600 (optical density at 600 nm) of 1 was reached. Subsequent cultivation was at 18 °C and an induction was done by supplying 1 mM IPTG (isopropyl-β-D-thiogalactopyranoside). After 6 hours 5 mg of the substrate cannabidiol (CBD isolate 99.034%, DB labs, Las Vegas, NV, United States) were added. In regular time intervals samples of 200 μL were taken from the culture. Cells were removed from samples by centrifugation and diluted 1:2 for HPLC-Analysis, controlling the progress of the biotransformation.

CBD-2βG was purified via adsorption to a polystyrene matrix and elution with methanol.

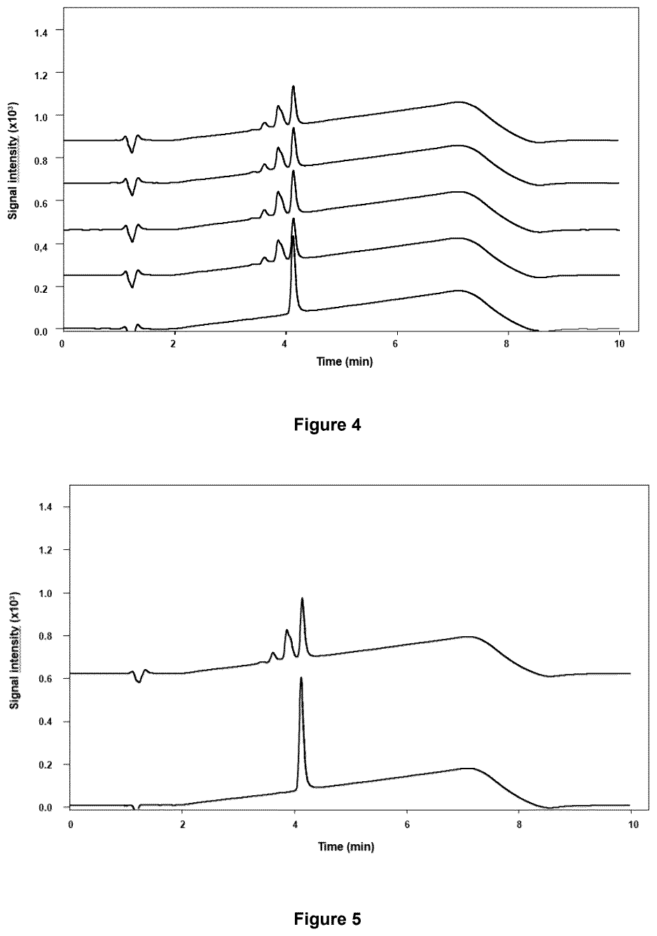

For the cyclodextringlucantransferase reaction each 0.0175 mg of CBD-2BG from a stem solution were incubated with 2.25 U CGTase (Amano) in 25 mM sodium acetate buffer pH 5.5 and with either 0.056 mg α-, β-, γ-cyclodextrin or starch from stem solutions in an end volume of 0.14 mL and were incubated overnight at room temperature. Aliquots of the respective reactions were analyzed by HPLC (FIG. 4).

### Example 2

### Synthesis of CBD-2βG-Derived Oligosaccharides

CBD-2βG, the intermediate product of Example 1, was used for a small preparative projected synthesis of CBD-2βG-8αG. For this, 5 mg of CBD-2βG were incubated with 450 U CGTase (Amano) in 25 mM sodium acetate buffer pH 5.5 with 15 mg γ-cyclodextrin in an end volume of 5 mL and were incubated overnight at room temperature. An aliquot of the respective reactions was analyzed by HPLC (FIG. 5).

### Example 3

### LC-MS-MS2-Analytics of CBD-2βG-Derived Oligosaccharides

Analytics by liquid chromatography coupled with mass spectroscopy were performed with a Bruker esquire 3000plus (Bruker Daltonics, Bremen, Germany) mass spectrometer with an Agilent 1100 HPLC system, an Agilent 1100/1200 Micro Wellplate Autosampler 1, and an Agilent 1100/1200 Diode Array Detector SL 1. For mass spectroscopy a Dual Ion Source (DUIS) was used and run in a negative mode of ionization.

The separation was performed with a reversed-phase column LUNA C18 100A 150×2 mm (Phenomenex, Aschaffenburg, Germany). As mobile phases Milli-Q purified water (A) and methanol (B), each with 0.1% formic acid, were used. The flow rate was 0.2 mL min⁻¹, the gradient was: 0 min 40% B, 15 min 100% B, 20 min 40% B, 30 min 40% B. The sample injection volume was 5 μL. Flow to mass spectroscopy was regulated by a flow separator to 0.2 mL. The mass spectroscopy profiles were recorded from mass/charge ratios of 50 m/z to 1000 m/z with negative ionization and a scan speed of 4000 V and an interface voltage of −500 V using nitrogen as collision gas.

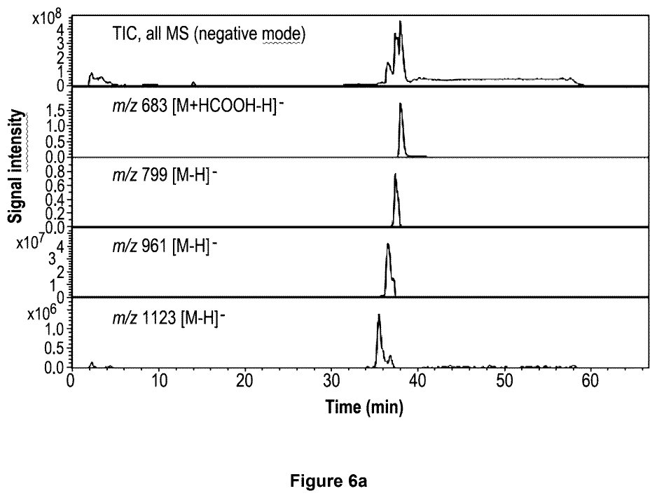

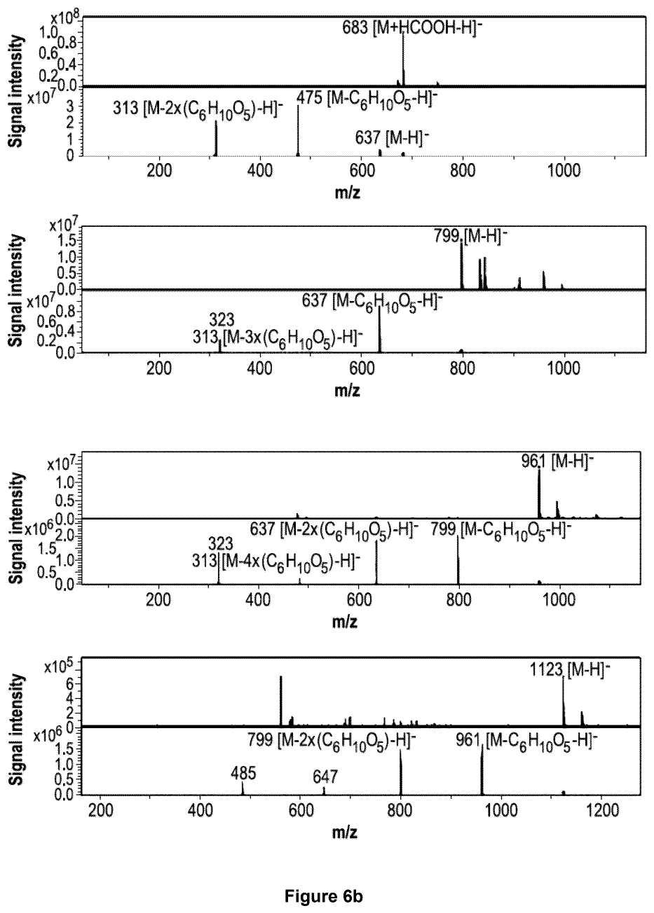

FIG. 3 shows a LC-MS analysis of the products of the biotransformation of cannabidiol to CBD-oligosaccharides of Example 2. Results show, that the staggered cannabinoid oligosaccharides CBD-2βG-1αG, CBD-2βG-2αG, and CBD-2βG-3αG have been obtained as a mixture (FIG. 6). Structure with respect to anomeric character of glucopyranosyl residues is further supported by hydrolytic cleavage experiments (Example 4).

### Example 4

Hydrolytic Cleavage of Staggered Oligosaccharides CBD-2βG-1αG, CBD-2βG-2αG, and CBD-2βG-3αG by saliva

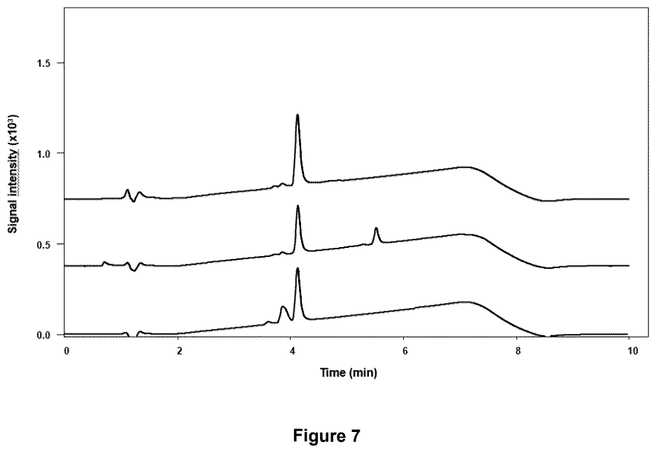

0.2 mL of a solution of 0.5 mg/mL staggered oligosaccharides CBD-2βG-1αG, CBD-2βG-2αG, CBD-2βG-3αG, and 0.5 mg/mL CBD-2βG (left-over substrate) from Example 2 were mixed with 0.1 mL saliva and incubated for 24 hours at 30 °C. The samples were analyzed by HPLC (FIG. 7 top graphic, control in bottom graphic).

The result demonstrates the faster hydrolyzation of the α-glucopyranosyl residues of this cannabinoid oligosaccharide composition with saliva in comparison to its β-glucopyranosyl stem glycosylation (left in this assay as CBD-2βG).

### Example 5

### Hydrolytic Cleavage of Staggered Oligosaccharides CBD-2βG-1αG, CBD-2βG-2αG, and CBD-2βG-3αG by ß-Glucosidase

0.2 mL of a solution of 0.5 mg/mL staggered oligosaccharides CBD-2βG-1αG, CBD-2βG-2αG, CBD-2βG-3αG and 0.5 mg/mL CBD-2βG (left-over substrate) from Example 2 were supplied with 5 mg Rapidase (a mixture of β-glucosidases) dissolved in 0.1 mL water and incubated for 24 hours at 30 °C. Control was with 0.1 mL water instead of enzyme solution. The samples were analyzed by HPLC (FIG. 7 middle graphic, control bottom graphic).

The result demonstrates the degradation of CBD-2βG at the stem glycosylation and removal of glucopyranosyl residues, respectively, are possible as foreseen in applications of the cannabiboid oligosaccharide compounds and compositions with release of the aglycon. The free aglycon is insoluble in water and not determinable with this assay, only the still water-soluble intermediate of degradation CBD-1βG is observable.

### Example 6

Formation of CBD-Oligosaccharides via CBD-2βG with Glucansucrases

Each 50 mL MRS medium were inoculated from stems of the glucansucrase (dextransucrase) forming strains Liquorilactobacillus hordei TM 1.1822 and Liquorilactobacillus nagelii TM 1.1827 and incubated over night at 35 °C and 200 rpm. Both cultures of each 50 mL were centrifugated for 15 min and 4.700 rpm. Each cell pellet was resuspended in 1 mL 100 mM citrate buffer pH 6 supplemented with 0.25 mg CBD-2βG and 10 mg sucrose and incubated at 30 °C and shaken with 300 rpm. At 1, 2, and 4 hours 0.2 mL samples for analysis were taken and centrifuged at 15.000 rpm for 15 min.

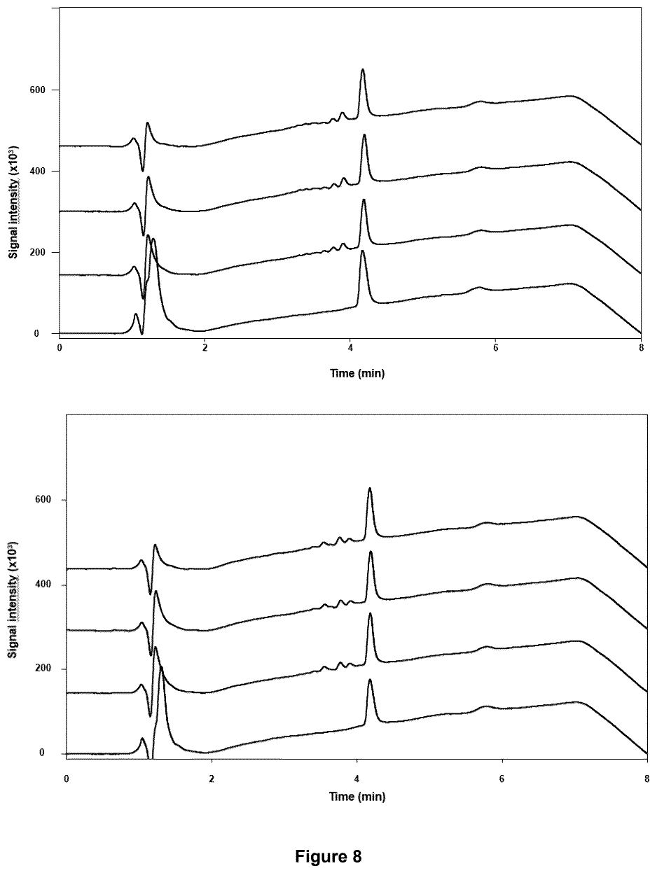

HPLC-analysis of both reaction mixtures shows the formation of staggered CBD-oligosaccharides from CBD-2βG by glucan (dextran) sucrases of Liquorilactobacillus hordei TM 1.1822 and Liquorilactobacillus nagelii TM 1.1827. The products were CBD-2βG-1αG, CBD-2βG-2αG, and CBD-2βG-3αG ((1-6)-α-linked) (FIG. 8); efficacy was lower than with CGTase resulting in α-(1-4)-linked products. From the known products specificity of Liquorilactobacillus hordei TM 1.1822 and Liquorilactobacillus nagelii TM 1.1827 as dextransucrases the link of α-glucopyranosyl residues must be α-(1-6), unlike those of CGTase products with α-(1-4) links.

1. A method for producing cannabinoid-oligosaccharide(s), optionally cannabidiol (CBD)-oligosaccharide(s), comprising the step of contacting a cannabinoid like CBD with an isolated UDP-carbohydrate-dependent glycosyltransferase preferably of the class UGT73A, ptionally with a glycosyltransferase encoded by the nucleic acid sequence of SEQ ID NO: 1 or having the amino acid sequence of SEQ ID NO:2 or a glycosyltransferase encoded by the nucleic acid sequence of SEQ ID NO:3 or having the amino acid sequence of SEQ ID NO:4, or with a UDP-carbohydrate-dependent glycosyltransferase, which has a nucleic acid sequence identity of at least 70%, of at least 80%, of at least 90%, of at least 95% or of at least 98% to SEQ ID NO:1 or an amino acid sequence identity of at least 70%, of at least 80% of at least 90%, or of at least 95% to SEQ ID NO:2, or with a UDP-carbohydrate-dependent glycosyltransferase, which has a nucleic acid sequence identity of at least 70%, of at least 80%, of at least 90%, of at least 95% or of at least 98% to SEQ ID NO:3 or an amino acid sequence identity of at least 70%, of at least 80% of at least 90%, or of at least 95% to SEQ ID NO: 4, in a reaction mixture under conditions, including any co-substrates and co-factors necessary for glucosyltransferase activity, effective to produce cannabinoid-glycoside(s), and of contacting this, subsequently or in parallel, or cannabinoid-β-D-glycosides from other sources, with a cyclomaltodextringlucanotransferase with at least one of its substrates cyclodextrin or substituted cyclodextrin (for example hydroxypropyl-, methyl-), linear maltodextrins, starch, or glycogen and another step of purifying the cannabinoid-oligosaccharide(s) from the reaction mixture to obtain the isolated cannabinoid-oligosaccharide(s).

2. The method of claim 1, wherein the isolated UDP-carbohydrate-dependent glycosyltransferase uses UDP-glucose or an activated form of fructose, glucose, galactose, mannose, rhamnose, ribose, arabinose, xylose, or an activated oligosaccharide, or an activated aldonic or uronic acid, for example gluconic acid, glucuronic acid or galacturonic acid, or N-substituted derivatives of said carbohydrates, for example 2-N-acetyl glucosamine, as co-substrate and a cannabinoid like CBD, and wherein a cyclomaltodextringlucanotransferase uses the intermediary product (or added educt from other sources) cannabinoid-β-D-glycoside as a substrate to form higher glycosylated oligosaccharides (the latter with α-anomeric glucopyranosyl residues) and the products are the respective cannabidiol-oligosaccharides with stem β-D-glycosidic structures and branching α-glucopyranosyl-oligomers.

3. The method of claim 1, wherein the isolated UDP-carbohydrate-dependent glycosyltransferase and the cyclomaltodextringlucanotransferase are obtained by heterologous expression in one or two organisms of species different from the origin species of the glycosyltransferase or the cyclomaltodextringlucanotransferase; optionally the conversion of cannabinoids to cannabinoid-oligosaccharide(s) relies on the cells supply of the co-substrate identified as UDP-glucose or as other activated carbohydrates and putatively on the cells supply or import of at least one of the substrates cyclodextrin or substituted cyclodextrin (for example hydroxypropyl-, methyl-), linear maltodextrins, starch, or glycogen, and takes place within the same heterologous organism(s), which may be optimized by genetic and/or gene technological means for the purpose.

4. The method of claim 1, wherein instead of or in addition to cyclomaltodextringlucanotransferase and its substrates (cyclodextrin or substituted cyclodextrin (for example hydroxypropyl-, methyl-), linear maltodextrins, starch, or glycogen) glucansucrase(s) and their substrate sucrose (or other compatible disaccharides) are used for the addition of (1-2)-, (1-3)-, (1-4)-, or (1-6)-linked α-D-glucopyranoside residues to produce staggered cannabinoid-oligosaccharides.

5. The method of claim 1, wherein cannabinoid-oligosaccharides can be obtained, optionally but not limiting wherein CBD-2ßG-8αG and/or higher reaction products or staggered cannabinoid-oligosaccharides are obtained from cannabidiol with GT40/UDP-glucose and with cyclomaltodextrin-glucanotransferase/γ-cyclodextrin or with glucansucrases and sucrose (or other disaccharides).

6. The cannabinoid-oligosaccharides obtained by processes according to claim 1 with a glycosylation pattern comprising none or one to four β-D-glycopyranosyl stem residue(s) and one or more additional ((1-4)-α-D-glucopyranosyl)_{5, 6, 7, or >7} (1-4)-α-D-glucopyranoside)ₙ oligosaccharide residues or staggered cannabinoid-oligosaccharides, the staggered cannabinoid-oligosaccharides also be derived by other glycosyltransferase or transglycosylation reactions than cyclomaltodextringlucanotransferase like with glucansucrases and in this case may be not only be (1-4)- but also (1-2)-, (1-3)-, or (1-6)-linked, the (1-4)-α-D-glucopyranoside residue(s) may be directly attached to the cannabinoid, to its β-D-glycopyranosyl residue(s), or, if more than one, may also be attached to each other.

7. The CBD-oligosaccharides obtained according to claim 6, comprising none or one to four β-D-glucopyranosyl stem residue(s) and one or more additional ((1-4)-α-D-glucopyranosyl)_{5, 6, 7, or >7} (1-4)-α-D-glucopyranoside)ₙ oligosaccharide residues or being staggered cannabinoid-oligosaccharides which can be (1-2)-, (1-3)-, (1-4)-, or (1-6)-α-linked.

8. The compounds CBD-2βG-6αG, CBD-(βG-6αG) 2, CBD-2βG-12αG, CBD-2βG-7αG, CBD-(βG-7αG) 2, CBD-2βG-14αG, CBD-2βG-8αG, CBD-(βG-8αG) 2, CBD-2βG-16αG, CBD-2βG-(6/7/8αG)ₙ, and staggered cannabinoid-oligosaccharides, the latter can be (1-2)-, (1-3)-, (1-4)-, or (1-6)-α-linked, or esters thereof, or acetal, or semiacetal derivatives thereof or an ester of the latter.

9. The method of claim 1, wherein strains of Escherichia coli or yeasts, optionally Saccharomyces cerevisiae or Pichia pastoris, are used as microorganisms for production.

10. The method of claim 1, wherein the UDP-carbohydrate-dependent glycosyltransferase and the cyclomaltodextringlucanotransferase and/or a glucansucrase are heterologously expressed in Cannabis sativa and cannabinoids like cannabidiol from the secondary metabolism of the plant are used as substrate to be converted to the respective cannabinoid-oligosaccharides and these can be obtained by this means.

11. The method of claim 1, wherein the UDP-carbohydrate-dependent glycosyltransferase is heterologously expressed in Cannabis sativa and wherein the glucose residues at the 2′ and 6′ hydroxy groups of a cannabinoid like cannabigerolic acid act as protective groups preventing or reducing metabolization and cyclization to tetrahydrocannabinol(s).

12. A composition obtained by methods of claim 1, comprising
cannabinoid-oligosaccharides, optionally cannabidiol-oligosaccharides,
components of the reaction medium, or
components of an organism, and/or
components of a culture medium and their degradation products;
the compositions optionally may contain non-converted cannabinoid substrates like cannabidiol and byproducts, derived from cannabinoid-oligosaccharides, optionally acetates with variable sites of substitutions at the glucopyranosyl residues.

13. A method by which cannabinoid-oligosaccharides obtained by a method of claim 1 are enriched and purified in a second step from resulting compositions and wherein extractions with organic solvents, optionally ethylacetate, as well as adsorption to a lipophilic solid phase, optionally at a polystyrol-matrix, are used for enrichment and purification and an organic solvent, optionally methanol, is used for elution from this matrix.

14. The method of claim 1, wherein the first step of obtaining the cannabinoid-oligosaccharides from the organism Cannabis sativa for subsequent enrichment and purification is an extraction with an aqueous phase, or a hot aqueous phase (water or steam), or a polar solvent, or a polar solvent mixture.

15. A kit according to claim 1 for production, purification, and analytics of cannabinoid-oligosaccharides, preferably cannabidiol-oligosaccharides, comprising the gene and/or the enzyme of [Seq 1] and a gene and/or enzyme of a cyclomaltodextringlucanotransferase and/or a glucansucrase in form of a DNA-sequence, or a codon-optimized DNA-sequence, or an amino acid sequence, or an isolated DNA, or a DNA in a vector or an expression vector, or the enzyme or a composition containing the enzyme, or a transgenic microorganisms or a transgenic plant, as well as specific means and instructions for performing the processes of production and purification and for analytics and for quality control of the products.

16. The cannabinoid-oligosaccharides according to claim 1, optionally cannabidiol-oligosaccharides, optionally substituted at glycosidic residues, optionally acetylated or peracetylated, for use as biologically active compounds, as medicine or veterinary medicine, which in comparison to the respective free cannabinoid can have an improved water solubility, can have a solubilizing or emulsifying effect on other glycosides of cannabidiol or other cannabinoids with lesser number of carbohydrate residues (optionally CBD-1βG, CBD-2βG) or on free cannabidiol or on other free cannabinoids, or on other components with low solubility within a composition, optionally on aroma compounds or other biologically active compounds in a beverage, a different taste or mouth feeling, a different uptake to the body, a changed metabolization, a changed activity, or a changed pharmacokinetic.

17. A composition comprising
cannabinoid-oligosaccharides, optionally cannabidiol-oligosaccharides; and
at least one glycosidase, optionally at least one ß-glucosidase, or a composition of glycosidases, optionally ß-glucosidases; and/or
microorganisms which produce glycosidases, optionally ß-glucosidases; and/or
amylase(s) and/or
encapsulated forms of single, some, or all said components of the composition for use as active pharmaceutical principle, medicine, or veterinary medicine.

18. A method using cannabinoid-oligosaccharides, optionally cannabidiol-oligosaccharides and/or substituted derivatives according to claim 16 in concentrated aqueous solution to obtain a concentrate, hereby obtaining an intermediate product for the purposes described in claim 16, which can be used advantageously for the production of the respective end product, which as such concentrates are more durable or by their lesser volume and lesser weight are better storable or transportable; such a concentrate shows an at least twofold concentration of cannabinoid-oligosaccharides or their derivatives in comparison to the concentration in the respective end product (for example a beverage), optionally are at least fivefold concentrations in the said concentrate.

19. The use of cannabinoid-oligosaccharides with mixed α- and β-glucopyranosyl structure for differential release of the aglyca relying on the organ-specific (for example mouth cavity, stomach, small and large intestine) presence of hydrolytic enzymes/enzymes with glycosidase activity (optionally various human amylases, optionally microbial β-glucosidases) for the purpose of targeted drug delivery.
